(19) United States
(12) Patent Application Publication
(10) Pub. No.: US 2004/0171690 A1
(43) Pub. Date: Sep. 2, 2004

(54) Use of protein essential aminoacids to treat amenorrhea and related disorders

(76) Inventors: Patrick Ammann, Troinex (CH); Rene Rizolli, Genthod (CH); Jean-Philippe Bonjour, Veyrier (CH)

Correspondence Address: NOVARTIS, CORPORATE INTELLECTUAL PROPERTY, ONE HEALTH PLAZA 430/2, EAST HANOVER, NJ, 07936-1080, US

(21) Appl. No.: 10/475,950

(22) PCT Filed: Apr. 25, 2002
(86) PCT No.: PCT/EP02/04615

(30) Foreign Application Priority Data
Apr. 26, 2001 (GB) 0110288.8

(51) Int. Cl. A61K 31/198

(52) U.S. Cl. 514/561; 514/564; 514/565; 514/567; 514/400; 514/419; 514/562

(57) ABSTRACT

Malnourishment leading to a decrease in body weight interferes with estrogen secretion in women, causing deleterious effects on bone density and on the menstrual cycle. The invention is based on the discovery that it is possible to reverse these metabolic effects of malnourishment by boosting protein intake, as whole protein or as a blend of essential amino acids. The proteins may be administered in the form of a dietary supplement, as a foodstuff, or as a component of a complete meal.

### FIELD OF THE INVENTION

This invention concerns methods of treatment and prevention of amenorrhea and associated medical disorders, and in particular dietary means of reversing these conditions.

### BACKGROUND OF THE INVENTION

There has been an alarming rise in recent years in the diagnosis of eating disorders such as anorexia nervosa and bulimia, particularly among young people. Self-starvation, which is frequently combined with strenuous exercise, can lead to a large deficit in energy intake. Another group of individuals who sometimes fail to consume enough calories to satisfy their energy requirements are athletes undergoing intense physical training. In these cases of severe malnourishment the body adapts by cutting back on non-essential metabolic processes in order to survive.

Among girls and women one of the most striking symptoms of having a low body weight due to severe weight loss or lack of weight gain is amenorrhea, i.e. the absence of a menstrual cycle due to suspension of ovulation. The decrease in energy intake observed in conditions of severe dietary restriction is considered to be the main cause of amenorrhea in these circumstances.

In the long term, amenorrhea is associated with very serious consequences on health. For example, there is a strong correlation between amenorrhea and fertility problems. Also, because of the low levels of circulating estrogen in the blood, bone mass acquisition is decreased during growth, and bone loss is induced during adulthood. Having a low body mass index (BMI) is a recognized risk factor for osteoporosis. In women with anorexia nervosa serum levels of osteocalcin, a marker of bone formation, are significantly decreased in comparison with the levels in age-matched healthy controls. It is common knowledge that women with amenorrhea are particularly at risk of suffering from osteoporosis and bone fractures in later life.

In order to avoid the personal distress caused by infertility and brittle bones, and also to limit the burden on medical service providers responsible for treating these disorders, there is an urgent need for treatments capable of rapidly restoring normal menstrual cycles in women with amenorrhea.

In the ideal case, the malnourished amenorrheic woman will of her own accord resume balanced eating patterns and a diet of sufficient caloric content. However, since anorexia nervosa and other under-eating/over-exercising disorders are chronic syndromes which may afflict a person over an entire lifetime this target is usually unrealistic. Sufferers frequently fail to comply with prevailing medical advice, which is to increase the caloric content of their diet. Similarly, athletes are often concerned about keeping their bodies in peak physical condition and therefore about minimizing body fat content by controlling calorie consumption.

Current clinical intervention generally takes the form of estrogen therapy to over-ride starvation-induced shutdown or irregularity in menstrual cycles. However, this is an extreme measure of dubious efficacy which is associated with adverse side effects, and moreover there is concern that estrogen may promote the development of cancer. Calcium and vitamin D supplements are also frequently prescribed for bone support in malnourished women with amenorrhea. However, such treatment cannot fully counteract the negative effects of sex hormone deficiency and low protein intake.

Our studies have yielded unprecedented experimental results indicating that dietary intervention can induce a rapid resumption in normal hormonal cycling and menstruation, and promote an increase in bone mineral density (BMD) and body weight in women experiencing weight loss-induced amenorrhea. The key to achieving these effects is a boost in dietary protein intake. Unexpectedly, a blend of essential amino acids is just as effective in this respect as intact dietary protein. Contrary to expectations, the desired effects are obtained irrespective of, and optionally to the exclusion of, any increase in overall energy consumption. This treatment method is therefore ideally suited to women with amenorrhea who are reluctant to increase their overall calorie intake, and especially those who are averse to fat or carbohydrate consumption.

Protein supplements or meals for treatment of weight loss-induced amenorrhea can be self-administered for extensive periods without risk or adverse side-effects, yet are extremely effective in preventing long-term damage to the body caused by sex hormone imbalance.

### SUMMARY OF THE INVENTION

According to a first aspect of the invention there is provided use of two or more essential amino acids in free form or in salt form in the manufacture of a medicament or nutritional formulation for the restoration of normal physiological levels of estrogen in a premenopausal woman suffering from malnourishment.

In another aspect of the invention there is provided use of two or more essential amino acids in free form or in salt form in the manufacture of a medicament or nutritional formulation for the prevention or treatment of amenorrhea, oligomenorrhea or erratic menstruation, especially when caused by malnourishment.

In a further aspect of the invention there is provided use of two or more essential amino acids in free form or in salt form in the manufacture of a medicament or nutritional formulation for the prevention or treatment of osteopenia or osteoporosis in a premenopausal woman suffering from amenorrhea, oligomenorrhea or erratic menstruation.

In a further aspect of the invention there is provided use of two or more essential amino acids in free form or in salt form in the manufacture of a medicament or nutritional formulation for the prevention or reversal of body weight loss and/or loss of muscle mass in a premenopausal woman suffering from amenorrhea, oligomenorrhea or erratic menstruation due to malnourishment.

In a further aspect of the invention there is provided use of protein in the manufacture of a nutritional formulation for the prevention or treatment of amenorrhea, oligomenorrhea or erratic menstruation in a premenopausal woman suffering from malnourishment, wherein said nutritional formulation comprises at least 20 en % protein and is in the form of a carbonated or non-carbonated soft drink, a juice, a sports drink, a milk drink, a milk-shake, a yoghurt drink, a smoothie, a soy-based drink, a soup, a cereal bar, a candy bar, a dairy bar, a snack-food, a breakfast cereal, a candy, a tab, a cookie, a cracker, chocolate, chewing-gum, or a dessert.

In another aspect of the invention there is provided use of protein in the manufacture of a complete formula diet or enteral feeding solution for the prevention or treatment of osteopenia or osteoporosis due to malnourishment in a premenopausal woman suffering from amenorrhea, oligomenorrhea or erratic menstruation, wherein said complete formula diet or enteral feeding solution comprises at least 20 en % protein.

In yet another aspect of the invention there is provided use of protein in the manufacture of a complete formula diet or enteral feeding solution for the prevention or treatment of amenorrhea, oligomenorrhea or erratic menstruation in a premenopausal woman suffering from malnourishment, wherein said complete formula diet or enteral feeding solution comprises at least 20 en % protein.

According to a further aspect of the invention there is provided a pharmaceutical or nutritional composition for preventing or treating osteopenia, osteoporosis, amenorrhea, oligomenorrhea or erratic menstruation comprising a blend of amino acids in free form or in salt form, wherein said amino acid blend consists of leucine, lysine, isoleucine, phenylalanine, valine, arginine, threonine, histidine and tryptophan.

### DETAILED DESCRIPTION OF THE INVENTION

The method of treatment claimed is applicable to undernourished or malnourished women in general. The term “women” as used here refers to premenopausal women and to girls who are on the verge of undergoing puberty (peri-pubertal) or who are post-pubertal. Since a significant proportion of women will undergo a self-imposed diet at some point in their lives, and are therefore potentially at risk from lowering of blood estrogen levels and its impact on calcium deposition in the bone, the target group of malnourished and undernourished women is large. A vast number of women in under-developed countries are also chronically undernourished or starving due to food shortages, and the treatment method of the invention provides a simple yet practical way of helping these women return to health.

Malnourished younger women (15 to 35 years old) are likely to benefit most from the treatment method of the present invention since it is at this stage of life that peak bone mass is achieved, and impaired bone densification in these vulnerable years increases the likelihood that the woman involved will suffer from osteoporosis in later life. Patients having bone densities greater than two standard deviations below age- and gender-matched normal means are urgently in need of treatments such as the one described herein to halt and preferably reverse bone loss. The years from 15 to 35 are also those in which a woman is most likely to wish to conceive, so it is desirable to minimize any negative impact on fertility caused by malnutrition.

For the reasons explained above amenorrheic or oligomenorrheic patients suffering from eating disorders such as anorexia nervosa and bulimia nervosa require urgent attention to avoid irreversible damage to their reproductive system and skeleton, and are prime targets for the method of treatment described herein. Others for whom these treatments are envisaged include very active women, and particularly dancers and endurance athletes. Women who are not under-weight but who are restricting, or intending to restrict, their energy intake for weight loss purposes are also suitable subjects for this treatment, as are vegetarians and vegans or any woman whose diet is not properly balanced to allow maintenance of a normal menstrual cycle.

Malnourishment may be a state of chronic undernourishment or starvation in which caloric intake repeatedly falls short of that needed to balance catabolism in the body, or may be due to an imbalance in the composition of the diet. Analysis of the diet by a nutritionist, dietician or other skilled person will reveal whether an individual is malnourished or at risk of becoming malnourished. In the context of the invention a state of malnourishment is considered to be indicated by low body weight and/or low body fat content and/or low serum leptin concentration. Low body weight can be defined as a BMI of less than 20, and particularly less than 18. A body fat content of less than about 15-17% is considered to be low. As there is a correlation between body fat content and leptin levels, a serum concentration of this hormone of less than 5 ng/ml, especially less than 1.85 ng/ml, is also an indicator that the treatment method of the invention should be commenced in order to prevent or curtail menstrual irregularities.

Amenorrhea is defined herein as an absence of menses, especially for greater than 6 months, in non-pregnant pre-menopausal women. Primary amenorrhea is a lack of menarche, i.e. menstruation has never occurred. Secondary (hypothalamic) amenorrhea is a cessation of menses after at least one period. Oligomenorrhea is defined as 3 or fewer menstrual bleeds per year for 2 or more years. The treatment method of the invention is especially applicable to amenorrhea (primary or secondary) or oligomenorrhea or erratic or irregular menstruation associated with low body weight/body fat content or rapid weight loss. One aim of the treatment method of the invention is to restore menstrual cycling to a frequency and degree of regularity which is considered normal for the population group to which the woman belongs. For the purposes of defining the invention, normal physiological levels of estrogen are deemed to be ≧30 pg/ml serum estradiol, 30 pg/ml being the lower limit at which ovulation will resume. Preferably the serum estradiol is restored to values within the range 100-700 pg/ml, and most preferably 200-400 pg/ml. If menstruation is only partially restored or is irregular, this can nevertheless be indicative of a partial recovery in circulating estrogen levels, with associated improvements in bone mineral density (BMD).

One of the greatest advantages of this improved method of treating amenorrhea is that, contrary to conventional belief, there is no absolute requirement to increase the overall energy content of the diet in order to achieve reversal of symptoms. In fact, simply increasing the proportion of protein relative to the other major components of the diet (fat and carbohydrate) while maintaining an isocaloric diet is a surprisingly effective way of restoring normal menstrual cycles. As a result, women who are averse to increasing their calorie intake, such as those suffering from anorexia nervosa and other eating disorders, are more likely to consent to this new improved treatment than to conventional treatments involving force-feeding. For present purposes alterations of no more than ±10%, especially ±5%, in the total caloric value of the diet are deemed to be insubstantial changes, i.e. the diet is isocaloric.

The Examples show that the loss in BMD observed in response to undernourishment, for example lack of protein, is significantly improved when the proportion of protein (casein or essential amino acid (EAA) blend) in the diet is increased. This result has tremendous medical implications, because it demonstrates for the first time that skeletal weakening associated with malnourishment in premenopausal women can be reversed by altering the composition of the diet, without the need for treatment with estrogen or other drugs. Presumably, the primary effect of protein supplementation is on restoration of estrogen secretion and the menstrual cycle, with consequent effects on bone architecture. The method of the invention is therefore effective to treat premenopausal women suffering from, or at future risk of suffering from, osteopenia and osteoporosis associated with malnourishment.

The medicaments or nutritional formulations of the invention may consist exclusively of protein, or alternatively may comprise other nutritional components in addition to protein. “Protein” or “proteinaceous material” is used here to refer to any form of digestible protein, peptides, single amino acids, mixtures of amino acids, and combinations thereof. Preferred proteins are milk protein, casein, whey and hydrolysates or peptides thereof, egg protein, vegetable protein such as soy protein, pea protein, rice protein or wheat protein, free amino acids, and mixtures thereof. Whenever amino acids are referred to this term is intended to include the L forms of free amino acids or any other isomer thereof, in hydrated or anhydrous form, and to encompass salts of any of these amino acids.

In particular, one or more, preferably at least two, of the essential amino acids are preferred to be included in the protein component of the composition of the invention. The essential amino acids (EAAs) are histidine, isoleucine, leucine, lysine, methionine, phenylalanine, threonine, tryptophan and valine. In one embodiment of the invention the protein component of the composition of the invention comprises at least 30% by weight, preferably at least 45%, branched chain amino acids (valine, isoleucine, leucine). The term “EAA blends” or “EAA compositions” is intended to refer to any composition comprising at least one EAA. The particular amino acid blend selected may vary according to the patient to be treated. For example, vegetarians or vegans may benefit most from products containing those amino acids which are lacking in plant material. Where the subject to be treated by the method of the invention is suffering from lack of appetite or unwillingness to eat, it may be advisable to omit tryptophan in free amino acid form from the composition because of the postulated link between serotonin levels and anorexia. Optionally, for humans it is possible to employ a mixture of all the essential amino acids minus methionine.

In a preferred embodiment of the invention, the protein included in the special diet of the invention comprises a blend of one or more amino acids selected from leucine, lysine, isoleucine, phenylalanine, valine, arginine, threonine, histidine and tryptophan, with or without additional protein. For example, the amino acids may be present in the following relative proportions by weight:

| | |
|---|---|
| leucine | 1.0-1.5 |
| lysine | 0.75-1.0 |
| isoleucine | 0.4-0.75 |
| phenylalanine | 0.35-0.6 |
| valine | 0.25-0.6 |
| arginine | 0.25-0.6 |
| threonine | 0.25-0.5 |
| histidine | 0.25-0.5 |
| tryptophan | 0-0.25 |

A particularly preferred formulation has the following amino acid composition: leucine 24-28%, lysine 14-18%, isoleucine 10-14%, phenylalanine 10-14%, valine 8-12%, arginine 6-8% 6-8%, histidine 6-8%, and tryptophan 2-3%, by weight, based on total weight of amino acids.

Optionally, the protein component of the composition of the invention consists exclusively of or essentially of any of the blends of free amino acids described herein. More usually, the protein component will comprise a mixture of whole protein and free amino acids, preferably in a weight ratio in the range 1:5 to 5:1. A formula product comprising whey protein in combination with a blend of essential amino acids may be used. In a preferred embodiment of the invention the protein component comprises whey protein and an amino acid blend in a weight ratio in the range of about 2:1 to 4:1, preferably about 3:1. The total protein content of the nutritional formulation or medicament of the invention is preferably at least 20 en % (energy %), or at least 25 en %, for example in the range 25-100 en %, preferably 30-90 en %, and most preferably 40-80 en %, based on the total calories in the nutritional formulation or medicament.

Optionally, the protein is provided in the form of a nutritionally balanced complete meal, which is suited for oral or tube feeding. A complete formula diet or complete meal fulfilling all nutritional requirements will comprise fat and carbohydrate in addition to protein, plus fiber (soluble and/or insoluble), and a range of minerals and vitamins. For formula diets, or indeed any other form of balanced nutritional product, the relative proportions in en % of protein:fat:carbohydrate are optionally in the ranges 25-35:8-30:40-65. It may be desirable to provide the protein in the form of a low calorie meal replacement or other nutritional product. In this case the meal replacement or other nutritional product is preferably low fat, i.e. <10 en %, or substantially fat-free, i.e. less than 2.5 en % contributed by fat, such as about 2 en % fat. Suitably, a single serving of a low calorie meal replacement will have a calorific value of less than 1000 kcal, and preferably between 200 kcal and 500 kcal.

Alternatively, the protein is provided as a dietary supplement to be included in the diet in quantities such that the target amount of protein or calorific contribution from protein is achieved.

The target caloric contribution of protein in the diet as a whole is preferably at least 25 en %, more preferably at least 30 en %.

In accordance with the dietary regimen of the invention it is intended that a woman will consume a total of at least log of protein per day, and preferably 15-200 g, most preferably 20-100 g per day.

The medicament or nutritional formulation of the invention may be administered under the supervision of a medical specialist, or may be self-administered.

For convenience the protein/amino acid-containing formulation may be provided in oral nutritional form as a complete meal, as a powder for dissolution, e.g. hot chocolate, health drinks, as a solution, as a ready-made drink, optionally low calorie, such as a soft drink, including juices, milk-shake, yoghurt drink, smoothie or soy-based drink, in a bar, or dispersed in foods of any sort, such as baked products, cereal bars, dairy bars, snack-foods, soups, breakfast cereals, muesli, candies, tabs, cookies, biscuits, crackers (such as a rice crackers), chocolate, and dairy products. For women who are physically active, especially those who over-exercise, such as sportswomen, dancers and anorexics, a sports drinks formulation is a very convenient way of delivering a protein supplement. Preferably the sports drinks formulation is isotonic and comprises electrolytes and a source of sugar(s) or artificial sweetener(s). Optionally, the formulation may be administered in the form of a tube feeding solution or intravenously.

In a preferred embodiment of the invention the protein component of the invention is administered in a pharmaceutical or nutritional composition also comprising one or more of calcium, magnesium, iron, zinc, phosphorus, vitamin D and vitamin K. A suitable daily amount is 0.1 mg to 3.6 g calcium, preferably 320 to 530 mg, and particularly preferred about 500 mg. In general, the daily dosage of vitamins and minerals in the nutritional formulation or medicament of the invention is 25-100% by weight of the dosages recommended by the health authorities, and most preferably 30-50%. Dietary fiber may also be a component of the compositions of the invention. For example, the fiber content may be in the range of 0 to 15%, preferably 2 to 10%, and most preferably 3 to 5%, by weight, based on the total weight of the composition. Further components of the supplement may include any bioactive compounds or extracts which are known to have health benefits, especially for building bone, for regulating hormonal imbalances, and/or for improving physical performance.

Conventional additives may be included in the compositions of the invention, including any of those selected from preservatives, chelating agents, effervescing agents, natural or artificial sweeteners, flavoring agents, coloring agents, taste masking agents, acidulants, emulsifiers, thickening agents, suspending agents, dispersing or wetting agents, antioxidants, and the like.

For pharmaceutical preparations or dietary supplements the protein may be compounded with pharmaceutically acceptable carriers, excipients or diluents in the forms of pills, tablets, coated or uncoated, hard or soft capsules, dragees, lozenges, oral solutions, suspensions and dispersion, syrups or sterile parenteral preparations. Suitable excipients include inert diluents such as calcium carbonate, sodium carbonate, lactose, calcium phosphate, sodium phosphate; granulating and disintegrating agents such as cornstarch or alginic acid; binding agents such as starch, gelatin or acacia; and lubricating agents such as magnesium stearate, stearic acid or talc. Encapsulation may be recommendable to mask the bitter taste when a large amount of free amino acids is present in oral dosage forms. Pharmaceutical preparations or dietary supplements can conveniently be manufactured so as to contain up to 75%, preferably 30-75% by weight protein, based on the total weight of the pharmaceutical preparation or dietary supplement.

For treatment of amenorrhea under clinical supervision it is possible to combine the nutritional approach with conventional pharmaceutical therapies such as estrogen replacement therapy or with appetite stimulants such as cannabis. For example, the composition of the invention may be provided in the form of a kit for separate, sequential or simultaneous administration in conjunction with estrogen or its analogues. The conventional pharmaceutical ingredient may conveniently be formulated together with protein in standard pharmaceutical dosage forms.

Optimally, the proteinaceous dietary supplement is consumed at least once a day on a regular basis until normal menstruation has resumed. When the protein supplement is supplied in the form of a food or beverage, a suitable serving size may be in the range 20 to 500 g, preferably 50 to 250 g. If provided in the form of a meal or in pharmaceutical form, one or several dosages of the protein-containing composition may be administered over a 24-hour period. Since these formulations are safe to consume, women who persist in manifesting the symptoms of eating disorders, or over-exercising, can continue taking these supplements for as long as required, and preferably until healthy eating patterns have been resumed.

### EXAMPLES

### Example 1

### Protein Supplementation can Restore Menstrual Cycling in Protein-Deprived Rats

To investigate the role of protein intake on sex hormone status, 6 month old adult female Sprague-Dawley rats were fed isocaloric diets based on rat chow (sugar, corn oil, caseinate, corn starch, fiber, vitamins and minerals etc.) containing 15, 7.5, 5.0 or 2.5% casein for a period of 15 weeks. The feed was kept isocaloric by adjusting the content of corn starch to compensate for changes in the amount of protein.

Estrogen peaks in rats for only a few hours per cycle (6 days), making it technically difficult to make a direct assessment of hormonal status. Consequently presence and duration of the menstrual cycle duration was evaluated indirectly by vaginal smear examination.

Measurement of plasma insulin-like growth factor 1 (IGF-1) was conducted by radioimmunoassay after extraction by acid-ethanol and cryoprecipitation using a kit from Nichols institute. Low IGF-1 levels are believed to correlate with low dietary protein intake, and thereby with reduced bone mass.

Dual energy X-ray absorptiometry (DXA) measurements were carried out using a Hologic QDR-1000 instrument adapted to measurement in small animals, using an ultra-high resolution mode (line spacing 0.254 mm and resolution 0.127 mm) and 0.9 mm diameter collimator. During the measurements the animals were anaesthetized with ketamine hydrochloride (100 mg/kg body weight).

After 12 weeks, rats fed 15, 7.5 or 5% casein containing diets had regular cycles of 5.6±0.2, 5.8±0.3, 6.1±0.3 days (means±SEM), respectively, but there was complete absence of cycle in rats fed a 2.5% casein diet.

At 15 weeks into the study, rats fed the 2.5% casein diet also exhibited significant reductions in bone mineral density (BMD) as measured by DXA at the proximal and midshaft tibia. There was also a significant loss of trabecular bone volume and number of trabeculae, with an increase in trabeculae spacing. Plasma IGF-I was markedly reduced by 3 weeks into the study.

In a follow-up experiment the effects of dietary protein supplementation were investigated on a group of rats which had previously been kept for 8 weeks on a low protein diet (2.5% casein) of the sort described above. The administration over 16 weeks of an isocaloric diet containing a total of 7.5% casein or 15% casein induced a restoration of cycles in approximately 60% and 80%, respectively, of the rats previously fed a 2.5% casein diet, as illustrated in Table 1.

| | | | | | |
|---|---|---|---|---|---|
| Casein | 15 | 7.5 | 2.5 | 2.5 + 5 | 2.5 + 12.5 |
| Content (%) | | | | | |
| Cycle | 6.4 ± 0.4 | 5.7 ± 0.7 | — | 5.9 ± 0.7 | 5.8 ± 0.4 |
| duration | | | | | |
| (days) | | | | | |
| No Cycle | 0 | 0 | 100 | 41 | 20 |
| (%) | | | | | |

In conclusion, the experiments have shown that a protein-deficient diet resulting in depressed sex hormone status can be compensated for by providing isocaloric protein supplements, which can restore normal menstrual cycles.

### Example 2

### Supplementation of the Diet with Essential Amino Acids can Restore Menstrual Cycling in Protein-Deprived Rats

In a study analogous to that described in Example 1, and using the same measurement protocols, adult female rats were fed isocaloric diets of rat chow having different protein compositions according to this protocol:

Group 1 (positive control): 15% casein in the form of calcium case mate (over 20 weeks)

Group 2 (negative control): 2.5% casein (over 20 weeks)

Group 3: 2.5% casein (over 12 weeks), followed by 2.5% casein +2.5% EAA blend (over 8 weeks)

Group 4: 2.5% casein (over 12 weeks), followed by 2.5% casein +5% EAA blend (over 8 weeks)

The EAA blend comprises:

| | % by weight |
|---|---|
| Amino acid | total EAA blend |
| leucine | 24.0 |
| lysine | 15.0 |
| isoleucine | 11.3 |
| phenylalanine | 10.9 |
| valine | 9.1 |
| DL-methionine | 7.4 |
| L-arginine | 6.8 |
| threonine | 6.6 |
| histidine | 6.5 |
| tryptophan | 2.4 |

An assessment of menstrual cycling by vaginal smears was made at 12 and at 20 weeks. The results are shown in Table 1. After sacrifice of the rats at 23 weeks the ovaries were dissected out and weighed.

TABLE 1
Sex Hormone status

| | | | Group 3 | Group 4 |
|---|---|---|---|---|
| | Group 1 | Group 2 | (2.5% | (2.5% |
| | (15% | (2.5% | casein + | casein + |
| | casein) | casein) | 2.5% EAA) | 5% EAA) |
| Number of rats | 7/7 | 2/7 | 1/8 | 0/8 |
| with cycles at | | | | |
| 12 weeks | | | | |
| Cycles duration | 5.4 ± 0.35 | * | * | * |
| (days) | | | | |
| *Insufficient | | | | |
| numbers for | | | | |
| statistical | | | | |
| analysis | | | | |
| Number of rats | 5/7 | 1/7 | 6/8 | 4/7 |
| with cycles at | | | | |
| 20 weeks | | | | |
| Cycles duration | 5.2 ± 0.20 | * | 4.7 ± 0.21 | 4.3 ± 0.25 |
| (days) | | | | |
| Ovary (mg) | 46.7 ± 4.2 | 25.5 ± 3.5 | 48.4 ± 3.0 | 46.0 ± 5.4 |

Very few rats (3 out of 23) fed a 2.5% casein diet showed evidence of cycling (assessed at 12 weeks). Subsequent supplementation of the low protein diet with the EAA blend restored cycling in a further 9 rats out of a group of 15 (assessed at 20 weeks). The weight of the ovaries was also preserved by EAA supplementation.

The rats were weighed at 0, 12 and 21 weeks. At 23 weeks the rats were slaughtered and the muscle weight was determined by dissecting out and weighing all the muscles surrounding the tibia.

TABLE 2
Body and Muscle Weight

| | | | | Group 3 | Group 4 |
|---|---|---|---|---|---|
| | | Group 1 | Group 2 | (2.5% casein + | (2.5% casein + |
| | Weeks | (15% casein) | (2.5% casein) | 2.5% EAA) | 5% EAA) |
| Body Weight | 0 | 318.9 ± 15.4 | 315.7 ± 10.2 | 318.1 ± 9.7 | 319.6 ± 9.0 |
| (g) | | | | | |
| | 12 | 301.0 ± 6.6 | 248.3 ± 7.0* | 245.0 ± 2.9* | 249.1 ± 9.1* |
| | 21 | 288.0 ± 4.6 | 221.1 ± 7.4* | 274.4 ± 4.6° | 278.9 ± 6.5° |
| Muscle | 23 | 5.47 ± 0.26 | 4.18 ± 0.21* | 5.55 ± 0.25° | 5.58 ± 0.27° |
| weight (g) | | | | | |

*statistically significant vs 15% casein
°statistically significant vs 2.5% casein

The loss in muscle weight and overall body weight caused by the low casein diet was reversed by EAA supplementation. At 21 and 23 weeks, respectively, there was no significant difference in overall body weight or muscle weight in Groups 3 and 4 compared with Group 1.

An assessment of bone mineral density (BMD) by DXA at the spine, proximal tibia and midshaft tibia was made at 0, 12 and 21 weeks. A decrease in BMD at all 3 positions in response to the low casein diet was seen to be partially reversible by EAA, as shown in Table 3.

TABLE 3
BMD measured by DXA (g/cm² ± SEM)

| | | | | Group 3 | Group 4 |
|---|---|---|---|---|---|
| | | Group 1 | Group 2 | (2.5% casein + | (2.5% casein + |
| | Weeks | (15% casein) | (2.5% casein) | 2.5% EAA) | 5% EAA) |
| SPINE | 0 | 0.2312 ± 0.006 | 0.2310 ± 0.006 | 0.2355 ± 0.006 | 0.2351 ± 0.006 |
| | 12 | 0.2299 ± 0.006 | 0.2134 ± 0.005 | 0.2179 ± 0.005 | 0.2155 ± 0.005 |
| | 21 | 0.2355 ± 0.007 | 0.1991 ± 0.004* | 0.2206 ± 0.005° | 0.2227 ± 0.005° |
| PROXIMAL | 0 | 0.3189 ± 0.004 | 0.3172 ± 0.004 | 0.3170 ± 0.003 | 0.3224 ± 0.005 |
| TIBIA | 12 | 0.3080 ± 0.004 | 0.2816 ± 0.004 | 0.2871 ± 0.004 | 0.2848 ± 0.006 |
| | 21 | 0.3069 ± 0.01 | 0.2614 ± 0.003* | 0.2955 ± 0.005*° | 0.2919 ± 0.002*° |
| MIDSHAFT | 0 | 0.2646 ± 0.006 | 0.2644 ± 0.003 | 0.2627 ± 0.002 | 0.2644 ± 0.004 |
| TIBIA | 12 | 0.2658 ± 0.004 | 0.2559 ± 0.003 | 0.2611 ± 0.003 | 0.2616 ± 0.004 |
| | 21 | 0.2611 ± 0.006 | 0.2340 ± 0.005* | 0.2522 ± 0.002*° | 0.2524 ± 0.004*° |

*statistically significant vs 15% casein
°statistically significant vs 2.5% casein

### Example 3

### A Powdered Nutritional Formulation Comprising an EAA Blend, Suitable for Consumption by Malnourished Women Suffering from Amenorrhea

| | approximate % |
|---|---|
| | by dry weight |
| whey protein | 21% |
| EAA mix* | 7% |
| Fat | 5% |
| (of which | (2%) |
| essential | |
| fatty acids) | |
| Carbohydrate | 58% |
| Fiber | 9% |

(plus trace amounts of vitamins and minerals)

The proportions of protein:fat:carbohydrate (en %) are 29:12:59

A suitable single serving size of this composition is 50-75 g, to be taken at least once per day.

| | |
|---|---|
| L-leucine | 26 |
| L-lysine | 16 |
| L-isoleucine | 12 |
| L-phenylalanine | 12 |
| L-valine | 10 |
| L-arginine | 7 |
| L-threonine | 7 |
| L-histidine | 7 |
| L-tryptophan | 3 |
| | 100% |

### Example 4

### Sports Bar Suitable for use in the Invention

40 g bar providing (per 100 g):

energy: 414 kcal

25.5 g protein

48.2 g carbohydrate

13.2 g fat

+50% of the recommended daily dosages of vitamins E, C, B1, B2, niacin, B6, folic acid, B12, biotin and pantothenic acid

### Example 5

### Powdered Sports Drinks Suited for use in the Invention

Very high protein formulation (373 kcal/100 g)

90 en % protein (caseinate)

5 en % fat

4.7 en % carbohydrate

+iron, zinc and vitamins E, C, B1, B2, niacin, B6, folic acid, B12, biotin and pantothenic acid

High protein formulation (372 kcal/100 g)

33 en % protein

2 en % fat

63.4 en % carbohydrate

+iron, zinc and vitamins E, C, B1, B2, niacin, B6, folic acid, B12, biotin and pantothenic acid

1. Use of two or more essential amino acids in free form or in salt form in the manufacture of a medicament or nutritional formulation for the restoration of normal physiological levels of estrogen in a premenopausal woman suffering from malnourishment.

2. Use of two or more essential amino acids in free form or in salt form in the manufacture of a medicament or nutritional formulation for the prevention or treatment of amenorrhea, oligomenorrhea or erratic menstruation, especially that caused by malnourishment.

3. Use of two or more essential amino acids in free form or in salt form in the manufacture of a medicament or nutritional formulation for the prevention or treatment of osteopenia or osteoporosis in a premenopausal woman suffering from amenorrhea, oligomenorrhea or erratic menstruation.

4. Use of two or more essential amino acids in free form or in salt form in the manufacture of a medicament or nutritional formulation for the prevention or reversal of body weight loss and/or loss of muscle mass in a premenopausal woman suffering from amenorrhea, oligomenorrhea or erratic menstruation due to malnourishment.

5. Use according to any of claims 1 to 4 wherein said two or more essential amino acids are selected from the group consisting of: leucine, lysine, isoleucine, phenylalanine, valine, arginine, threonine, histidine and tryptophan.

6. Use according to any of claims 1 to 5 wherein the medicament or nutritional formulation comprises a blend of amino acids in free form or in salt form, in the following relative proportions by weight:

| | |
|---|---|
| leucine | 1.0-1.5 |
| lysine | 0.75-1.0 |
| isoleucine | 0.4-0.75 |
| phenylalanine | 0.35-0.6 |
| valine | 0.25-0.6 |
| arginine | 0.25-0.6 |
| threonine | 0.25-0.5 |
| histidine | 0.25-0.5 |
| tryptophan | 0-0.25 |

7. Use of protein in the manufacture of a nutritional formulation for the prevention or treatment of amenorrhea, oligomenorrhea or erratic menstruation in a premenopausal woman suffering from malnourishment, wherein said nutritional formulation comprises at least 20 en % protein and is in the form of a carbonated or non-carbonated soft drink, a juice, a sports drink, a milk drink, a milk-shake, a yoghurt drink, a smoothie, a soy-based drink, a soup, a cereal bar, a dairy bar, a snack-food, a breakfast cereal, a candy, a tab, a cookie, a cracker, chocolate, chewing-gum, or a dessert.

8. Use according to any of claims 1 to 7 wherein said malnourishment is caused by an eating disorder, overexercise and/or starvation.

9. Use according to any of claims 1 to 7 wherein said nutritional formulation or medicament is provided as part of a planned weight loss program.

10. Use according to any of claims 1 to 7 wherein said nutritional formulation or medicament is provided as nutritional support for physical training.

11. Use of protein in the manufacture of a complete formula diet or enteral feeding solution for the prevention or treatment of osteopenia or osteoporosis due to malnourishment in a premenopausal woman suffering from amenorrhea, oligomenorrhea or erratic menstruation, wherein said complete formula diet or enteral feeding solution comprises at least 20 en % protein.

12. Use of protein in the manufacture of a complete formula diet or enteral feeding solution for the prevention or treatment of amenorrhea, oligomenorrhea or erratic menstruation in a premenopausal woman, especially a woman suffering from malnourishment, wherein said complete formula diet or enteral feeding solution comprises at least 20 en % protein.

13. A pharmaceutical or nutritional composition for preventing or treating osteopenia, osteoporosis, amenorrhea, oligomenorrhea or erratic menstruation comprising a blend of amino acids in free form or in salt form, wherein said amino acid blend consists of leucine, lysine, isoleucine, phenylalanine, valine, arginine, threonine, histidine and tryptophan.

14. A pharmaceutical or nutritional composition according to claim 13 wherein the composition comprises said amino acids in the following relative ratios by weight:

| | |
|---|---|
| leucine | 1.0-1.5 |
| lysine | 0.75-1.0 |
| isoleucine | 0.4-0.75 |
| phenylalanine | 0.35-0.6 |
| valine | 0.25-0.6 |
| arginine | 0.25-0.6 |
| threonine | 0.25-0.5 |
| histidine | 0.25-0.5 |
| tryptophan | 0-0.25 |

15. A pharmaceutical or nutritional composition according to claim 13 having the following amino acid composition, based on total weight of amino acids: leucine 24-28%, lysine 14-18%, isoleucine 10-14%, phenylalanine 10-14%, valine 8-12%, arginine 6-8%, threonine 6-8%, histidine 6-8%, and tryptophan 2-3%.

16. A pharmaceutical or nutritional composition according to any of claims 13 to 15 and further comprising one or more additional active ingredients selected from the group consisting of: iron, zinc, calcium, magnesium, phosphorus, vitamin D and vitamin K.

17. A pharmaceutical or nutritional composition according to any of claims 13 to 16 which further comprises casein and/or whey protein.

18. A method of treatment to restore normal physiological levels of estrogen in a pre-menopausal woman suffering from malnourishment and in need of such treatment, comprising altering the diet of said woman by increasing the proportion of protein relative to the sum of carbohydrate and fat, as a percentage of total calories, in the diet, or administering a nutritional formulation or medicament comprising protein in an amount such that the total protein content of the diet is at least 20 en %, or at least 25 en %, preferably at least 30 en %.

19. A method for preventing or treating amenorrhea, oligomenorrhea or erratic menstruation in a pre-menopausal woman, particularly a woman suffering from malnourishment, comprising altering the diet of said woman by increasing the proportion of protein relative to the sum of carbohydrate and fat, as a percentage of total calories, in the diet, or administering a nutritional formulation or medicament comprising protein in an amount such that the total protein content of the diet is at least 20 en %, or at least 25 en %, preferably at least 30 en %.

20. A method of preventing or treating osteopenia or osteoporosis, in a pre-menopausal woman suffering from amenorrhea, oligomenorrhea or erratic menstruation, especially that due to malnourishment, comprising altering the diet of said woman by increasing the proportion of protein relative to the sum of carbohydrate and fat, as a percentage of total calories, in the diet, or administering a nutritional formulation or medicament comprising protein in an amount such that the total protein content of the diet is at least 20 en %, or at least 25 en %, preferably at least 30 en %.

21. A method of preventing or reversing body weight loss and/or loss of muscle mass in a pre-menopausal woman suffering from amenorrhea, oligomenorrhea or erratic menstruation due to malnourishment, comprising altering the diet of said woman by increasing the proportion of protein relative to the sum of carbohydrate and fat, as a percentage of total calories, in the diet, or administering a nutritional formulation or medicament comprising protein in an amount such that the total protein content of the diet is at least 20 en %, or at least 25 en %, preferably at least 30 en %.

22. A method according to any preceding claim wherein the proportion of protein in said diet is increased to at least 25 en %, optionally at least 30 en %.

23. A method according to any of claims 18 to 22 wherein said protein or said nutritional formulation or medicament comprises, or consists of, one or more essential amino acids in free form or in salt form.

24. A method according to claim 23 wherein the protein or said nutritional formulation or medicament comprises, or consists of two or more essential amino acids in free form or in salt form.

25. A method according to any of claims 18 to 24 wherein said malnourishment is caused by an eating disorder, overexercise or starvation.

26. A method according to any of claims 18 to 25 wherein said woman has a BMI of less than 20 and/or a body fat content of less than 17% and/or a serum leptin concentration of less than 5 ng/ml.

27. A method according to any of claims 18 to 26 wherein the overall caloric value of the diet is substantially unchanged.
